(19) United States
(12) Patent Application Publication
(10) Pub. No.: US 2017/0073246 A1
(43) Pub. Date: Mar. 16, 2017

(54) WATER SOFTENER WATER HARDNESS AUTOMATIC DETECTION DEVICE

(71) Applicant: Xiamen Runner Industrial Corporation, Xiamen (CN)

(72) Inventors: Yu Wei LIAN, Xiamen (CN); Feng Shun ZHAN, Xiamen (CN)

(21) Appl. No.: 15/261,056

(22) Filed: Sep. 9, 2016

(30) Foreign Application Priority Data
Sep. 16, 2015 (CN) 201520715431.6

(51) Int. Cl. C02F 1/00; G01N 33/18; C02F 5/00

(52) U.S. Cl. CPC C02F 1/008; C02F 5/00; G01N 33/1853; C02F 2201/005; C02F 2209/10

(57) ABSTRACT

A water softener water hardness automatic detection device includes: a water input tube; a water output tube; a valve head, disposed in a water softener; a bypass valve, disposed in a water softener, and connected to the valve head through the water input tube and the water input tube; and a water hardness detection device, disposed on the water input tube, used to detect hardness of water. The water hardness detection device is a TDS (total dissolved solids) detection probe, extending into a tube chamber of the water input tube, and connected electrically to a display panel of the water softener through a controller. The valve head and the bypass valve are both connected electrically to the controller. The water softener water hardness automatic detection device is simple in construction, user may adjust parameters of water softener, to achieve the most of its functions.

### BACKGROUND OF THE INVENTION

Field of the Invention

The present invention relates to a water hardness detection device, and in particular to a water softener water hardness automatic detection device.

The Prior Arts

Presently on the market, the water softener requires manual operation to detect ppm (parts per million) value of raw water, then it has to make further calculations to obtain hardness of water. As such, the water softener is not capable of detecting hardness of water automatically, while the user is not able to know the variations of water hardness, thus the effectiveness of water softener can not be fully realized.

Therefore, presently, the design and performance of the water softener is not quite satisfactory, and it leaves much room for improvement.

### SUMMARY OF THE INVENTION

In view of the problems and drawbacks of the prior art, the present invention provides a water softener water hardness automatic detection device, to overcome the shortcomings of the prior art.

The present invention provides a water softener water hardness automatic detection device, comprising: a water input tube; a water output tube; a valve head; a bypass valve; and a water hardness detection device. The valve head is disposed in the water softener. The bypass valve is also disposed in the water softener, and is connected to the valve head through the water input tube and the water input tube. The water hardness detection device is disposed on the water input tube, to detect hardness of water.

In an aspect of the present invention, the water hardness detection device is a TDS (total dissolved solids) detection probe, extending into a tube chamber of the water input tube, and connected electrically to a display panel of the water softener through a controller.

In another aspect of the present invention, the valve head and the bypass valve are both connected electrically to the controller.

Compared with the existing technology, the present invention has the advantages in that, it is simple in construction, and is adapted to realize the automatic detection of water hardness for the water softener, so that a user may know instantly the variations of water hardness. As such, the user may adjust related parameters of water softener, to achieve the most of its functions.

Further scope of the applicability of the present invention will become apparent from the detailed descriptions given hereinafter. However, it should be understood that the detailed descriptions and specific examples, while indicating preferred embodiments of the present invention, are given by way of illustration only, since various changes and modifications within the spirit and scope of the present invention will become apparent to those skilled in the art from the detailed descriptions.

### BRIEF DESCRIPTION OF THE DRAWINGS

The related drawings in connection with the detailed descriptions of the present invention to be made later are described briefly as follows, in which:

FIG. 1 is a top view of a water softener water hardness automatic detection device according to an embodiment of the present invention; and

FIG. 2 is a side view of a water softener water hardness automatic detection device according to an embodiment of the present invention.

### DETAILED DESCRIPTION OF THE PREFERRED EMBODIMENT

The purpose, construction, features, functions and advantages of the present invention can be appreciated and understood more thoroughly through the following detailed descriptions with reference to the attached drawings.

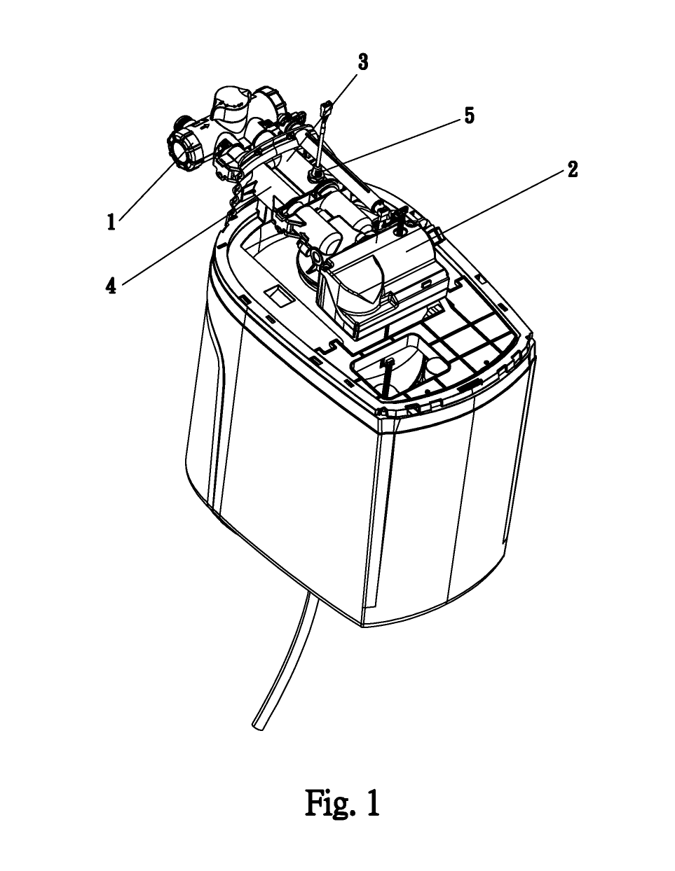

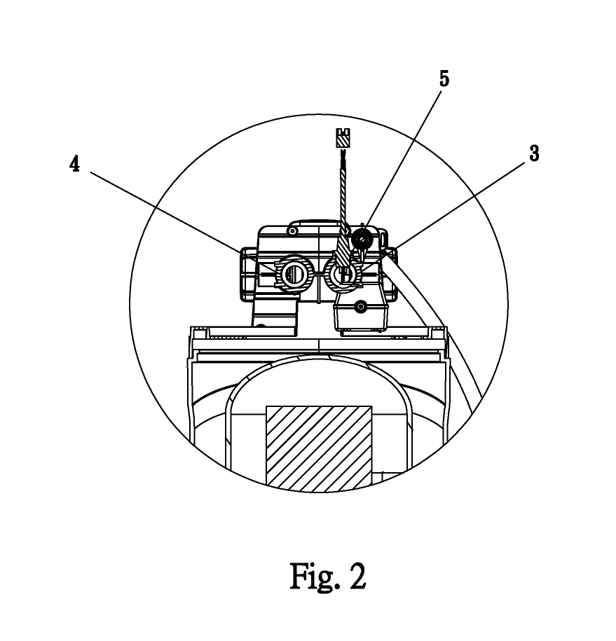

Refer to FIGS. 1 and 2 respectively for a top view of a water softener water hardness automatic detection device according to an embodiment of the present invention; and a side view of a water softener water hardness automatic detection device according to an embodiment of the present invention.

As shown in FIGS. 1 and 2, the present invention provides a water softener water hardness automatic detection device, comprising: a water input tube 3; a water output tube 4; a valve head 2; a bypass valve 1; and a water hardness detection device 5. The valve head 2 is disposed in the water softener. The bypass valve 1 is disposed in the water softener, and is connected to the valve head 2 through the water input tube 3 and the water input tube 4. The water hardness detection device 5 is disposed on the water input tube 3, to detect hardness of water.

In an embodiment of the present invention, the water hardness detection device 5 is a TDS (total dissolved solids) detection probe, extending into a tube chamber of the water input tube 3, and connected electrically to a display panel of the water softener through a controller.

In another embodiment of the present invention, the valve head 2 and the bypass valve 3 are both connected electrically to the controller.

The implementations of the present invention are as follows: in operation, in the water softener, raw water is input through the water input end of the bypass valve 1, and then it flows to the valve head 2 through the water input tube 3. At this time, the TDS detection probe 5 disposed on the water input tube 3 is used to detect PPM value of the input raw water, and water hardness value is displayed on the display panel, after the water is processed by the controller. As such, through controlling the valve head 2, water of various hardness can be output from the output port, in achieving maximum utilization of the water softener.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A water softener water hardness automatic detection device, comprising:
a water input tube;
a water output tube;
a valve head, disposed in a water softener;
a bypass valve, disposed in the water softener, and connected to the valve head through the water input tube and the water input tube; and
a water hardness detection device, disposed on the water input tube, to detect hardness of water.

2. The water softener water hardness automatic detection device as claimed in claim 1, wherein the water hardness detection device is a TDS (total dissolved solids) detection probe, extending into a tube chamber of the water input tube, and connected electrically to a display panel of the water softener through a controller.

3. The water softener water hardness automatic detection device as claimed in claim 1, wherein the valve head and the bypass valve are both connected electrically to the controller.
